(19) United States
(12) Patent Application Publication
(10) Pub. No.: US 2020/0147306 A1
(43) Pub. Date: May 14, 2020

(54) METHODS FOR TREATING CONGENITAL HYPERINSULINISM

(71) Applicant: XERIS PHARMACEUTICALS, INC., CHICAGO, IL (US)

(72) Inventors: STEVEN PRESTRELSKI, SAN DIEGO, CA (US); JOHN KINZELL, AUSTIN, TX (US); BRETT NEWSWANGER, AUSTIN, TX (US); PAUL THORNTON, FORT WORTH, TX (US); POUL STRANGE, PRINCETON JUNCTION, NJ (US); MARTY CUMMINS, GEORGETOWN, TX (US)

(21) Appl. No.: 16/630,774

(22) PCT Filed: Jul. 14, 2018
(86) PCT No.: PCT/US2018/042197

§ 371 Date: Jan. 13, 2020

Related U.S. Application Data
(60) Provisional application No. 62/532,856, filed on Jul. 14, 2017.

(51) Int. Cl. A61M 5/172; A61K 38/26; A61K 31/7004; A61K 38/28; A61P 3/10; A61M 5/14

(52) U.S. Cl. CPC A61M 5/1723; A61K 38/26; A61K 31/7004; A61K 38/28; A61P 3/10; A61M 2202/07; A61M 2230/201; A61M 2205/50; A61M 2205/581; A61M 2205/583; A61M 2205/18; A61M 5/1409

(57) ABSTRACT

A method of treating congenital hyperinsulinism in a subject is disclosed. The method can include parenterally administering to the subject a first composition comprising a glucagon, a glucagon analogue, or a salt form of either thereof, and optionally administering to the subject a second composition comprising glucose, a glucose analogue, or a salt form of either thereof, wherein administration of the first composition sufficiently increases blood glucose level in the subject such that the second composition is not administered or the second composition is administered at a glucose infusion rate (GIR) of less than 8 mg/(kg*min).

### CROSS-REFERENCE WITH RELATED APPLICATIONS

This Application claims priority to U.S. Provisional Patent Application Ser. No. 62/532,856 filed Jul. 14, 2017, which is incorporated herein by reference in its entirety.

### GOVERNMENT SPONSORED RESEARCH

This invention was made with government support under Grant Number 1 R 44 DK 105691-01 awarded by National Institute of Diabetes and Digestive and Kidney Diseases (NIDDK), National Institute of Health. The government has certain rights in the invention.

### BACKGROUND

### 1. Field of Invention

The present invention relates generally to glucagon delivery systems that can be used in combination with continuous glucose infusion therapy. In particular, the invention concerns the use of glucagon delivery systems that can be used to reduce or eliminate the need for glucose infusion therapy.

### 2. Description of Related Art

Patients with congenital hyperinsulinism (CHI) have a genetic defect which causes their pancreatic beta cells to over express insulin. This leads to severe hypoglycemia, which can cause seizures, coma and death. Untreated, frequent severe episodes of hypoglycemia lead to profound neurological impairment in many people, especially children and more especially neonates, with CH.

Symptoms of hypoglycemia vary greatly among patients, but typically include tremor, palpitations, irritability, anxiety, nervousness, hunger, tachycardia, headache and pallor. The symptoms typically subside once plasma glucose is restored to normal levels. If hypoglycemia is not reversed, a further decrease in plasma glucose can lead to depletion of glucose in the central nervous system and associated neuroglycopenic symptoms, such as difficulty in concentration, slurred speech, blurred vision, reduction in body temperature, behavioral changes and, if not treated, unconsciousness, seizure and possibly death.

In general, hypoglycemia can be defined as minor to moderate hypoglycemia or as severe hypoglycemia as follows:

Minor to Moderate Hypoglycemia:

Episodes that the patient can self-treat, regardless of the severity of symptoms, or any asymptomatic blood glucose measurements in which blood glucose levels are less than 70 mg/dL (3.9 mmol/L) and greater than 50 mg/dL (2.8 mmol/L).

Severe Hypoglycemia:

Operationally defined as an episode of hypoglycemia that the patient cannot self-treat so that external help is required. Typically, neuroglycopenic symptoms and cognitive impairment begin at a blood glucose level of about 50 mg/dL (2.8 mmol/L) and less.

Current treatments for CHI include administering drugs such as diazoxide or octreotide to block insulin release from the pancreas, but these have significant side effects and are effective in less than half of all cases. Therefore, a preferred treatment modality for CHI is continuous infusion of 50% dextrose (D50), i.e., D-glucose. Because of the high glucose infusion rate (GIR) that is required to treat CHI, D50 is normally delivered via a peripherally inserted central catheter, or PICC line, which must be implanted surgically. The PICC line is a source of infection for the patient, and a high GIR can cause fluid overload, which can lead to heart failure, pulmonary edema, and cyanosis.

A goal of CHI therapy is to reduce GIR requirement to the point where the PICC line can be removed in favor of safer IV administration of D50. This point is generally a GIR of less than 8 mg/(kg*min). Unfortunately, a cost effective and/or long term solution to achieve this goal is currently lacking.

Congenital hyperinsulinism (CHI) arises from dysregulated insulin secretion and is characterized by severe hypoglycemia (defined as blood glucose ≤70 mg/dL) due to inappropriately high blood insulin levels. Infants afflicted with persistent hyperinsulinemic hypoglycemia have variable long-term outcomes, depending on the successful ability to maintain euglycemia (blood glucose levels between 70 and 180 mg/dL) and thus avoid the elevated risk of permanent brain damage associated with hypoglycemic episodes. The cause of neonatal-onset CHI varies, and can require intensive surgical or medical care, such as surgical excision of the effected region of the pancreas (i.e. subtotal or near-total pancreatectomy). However, apart from being costly and invasive, a pancreatectomy (both subtotal and even near-total) does not ensure successful treatment in all patients, and greatly increases the risk of Type II diabetes mellitus and pancreatic exocrine insufficiency later in life.

Multiple drugs are also used to attempt to maintain euglycemia in CHI patients. One example is diazoxide, which was introduced in the mid-1960s, but this drug is only effective in a subgroup of CHI patients, particularly those whose condition arises from a mutation in the sulfonylurea receptor. Additionally, octreotide can also be used to block insulin secretion from the pancreas, but as with diazoxide it is also only effective in a subgroup of patients. The main treatment modality for CHI is continuous infusion of glucose, for example as 50% (w/v) Dextrose (D50; 50% Dextrose (D50) is typically available as a 0.5 g/mL dextrose aqueous solution. Alternative concentrations of dextrose are also available, for example D60 (60% (w/v) dextrose aqueous solution). Because of the high glucose infusion rate (GIR) that is required to treat CHI, the glucose infusion (e.g. via D50) is normally delivered via a peripherally inserted central catheter, or PICC line, which must be implanted surgically. The PICC line is a source of infection for the patient and a high GIR can cause fluid overload, which can lead to heart failure, pulmonary edema, and cyanosis. The primary goal of CHI therapy is to reduce the GIR requirement (e.g. <8 mg/(kg*min)), at which point the PICC line can be removed in favor of safer IV administration of D50.

An alternative treatment that has been proposed is glucagon infusion to increase blood glucose levels via stimulation of hepatic glycogenolysis. The rationale for this treatment stems from the reported observation that the high insulin levels in CHI patients inhibits glycogenolysis, thereby increasing the glycogen content stores in the body. The introduction of exogenous glucagon will promote glycogenolysis and help maintain blood glucose levels in the euglycemic range. Moreover, diminished glucagon serum levels have been reported in CHI patients during episodes of hypoglycemia, indicating that introduction of exogenous glucagon may be necessary to stimulate glycogenolysis.

However, while the concept of delivering exogenous glucagon via continuous infusion has been proposed, its practice in the clinic has been hampered by the inability to prepare a stable and soluble liquid glucagon formulation. Glucagon, specifically aqueous glucagon, has a widely-reported propensity to fibrillate and form insoluble aggregates that can clog the infusion lines and prevent dose delivery. A published study that attempted subcutaneous delivery of a glucagon solution reported frequent catheter obstruction, with incidents occurring daily or 2 to 3 times per week (Mohnike et al. 2008).

Accordingly, there remains a need for a stable glucagon formulation that may be administered via continuous subcutaneous infusion through a pump-based system (e.g. a no-loop system such as a patch-pump) that will not clog the infusion line, and thus ensure complete delivery of the dose to the patient. Such a treatment would allow the GIR to be sufficiently lowered such that the PICC line may be removed from the patient, and allow effective treatment of the disorder and thereby avoid the cost and complications associated with subtotal and near-total pancreatectomy.

### SUMMARY OF THE INVENTION

A solution to the current problems associated with treating CHI in patients has been discovered. The solution is premised on using stable and flowable glucagon formulations in combination with pump-based delivery systems to reduce GIR requirement in CHI patients, at which point a PICC line can be removed in favor of safer IV administration of D50. In particular, a soluble glucagon formulation can be delivered as a continuous subcutaneous infusion (CSI) via a pump system, e.g., a patch-pump system, to counter-act the overexpression of insulin in children with CHI. CSI glucagon can be added to existing glucose infusion therapy (e.g. using D50) with the expectation that blood sugar levels will rise, reducing or eliminating the need for glucose infusion therapy. It is believed that use of CSI glucagon can result in a 33% reduction in GIR in treated subjects. It is also believed that use of CSI glucagon can reduce GIR below the critical value of 8 mg/(kg*min) so that a PICC line can be safely removed. In the context of the disclosed invention, CH and CHI can be used interchangeably with congenital hyperinsulinism throughout the specification.

In one aspect of the present invention, there is disclosed a method of treating congenital hyperinsulinism in a subject. The method can include: (a) parenterally administering to the subject a first composition comprising a glucagon, a glucagon analogue, or a salt form of either thereof; and (b) optionally administering to the subject a second composition comprising glucose, a glucose analogue, or a salt form of either thereof. In certain instances, administration of the first composition sufficiently increases blood glucose level in the subject such that the second composition is not administered or the second composition is administered at a glucose infusion rate (GIR) of less than what would otherwise be needed had the subject not being administered the first composition. The composition can be a flowable composition. The flowable composition can be a solution, aqueous or non-aqueous. In certain aspects, the GIR is less than 20, 19, 18, 17, 16, 15, 14, 13, 12, 11, 10, 9, 8, 7, 6, 5, 4, 3, 2, or 1 mg/(kg*min). The GIR can be any range or number therein. In some aspects the GIR can be at least 33% less than what would otherwise be needed had the subject not been administered the first composition. The second composition can be intravenously administered to the subject. The subject can be currently undergoing or has previously undergone glucose injection at a second GIR prior to step (a), and wherein the second GIR is greater than the first GIR. In certain aspects, the glucose injection prior to step (a) is being or has been administered through a peripherally inserted central catheter. The second composition can have an aqueous solution comprising 5% (w/w) to 60% (w/w) d-glucose. In certain aspects, the second composition has about 50% (w/w) d-glucose or has about 10% (w/w) d-glucose.

Pump-based systems of the present invention that can be used to administer the glucagon compositions can be closed-loop, open-loop, or no-loop systems. The glucagon formulations that can be used with such systems are designed to be carried or stored in a pump container without having to be reconstituted (i.e., they are readily available to be administered to the patient from the pump container). Further, the formulations are stable at non-refrigerated temperatures (20-35° C.) for extended periods (>2 months) (i.e., the formulations can be safely stored in the pump container without risking substantial loss in activity of the glucagon in the formulation or risking the formation of insoluble aggregates than will inhibit delivery and clog the infusion apparatus).

The pump-based system can include: (1) a glucose sensor that is or can be inserted in a patient and that is capable of measuring blood glucose levels (e.g., either directly via contact with the patient's blood or indirectly via contact with the patient's interstitial fluid); (2) a transmitter that sends the glucose information from the sensor to a monitor (e.g., via radio frequency transmission); (3) a pump that is designed to store and deliver the glucose formulation to the patient; and/or (4) a monitor (e.g., one that can be built into the pump device or a stand-alone monitor) that displays or records glucose levels. For a closed-loop system, the glucose monitor can be capable of modifying the delivery of the glucagon formulation to the patient via the pump based upon an algorithm. Such a closed-loop system requires little to no input from the patient and instead actively monitors blood glucose levels and administers the needed amount of the glucagon formulation to the patient to maintain an appropriate glucose level and prevent the occurrence of hypoglycemia. For an open-loop system, the patient would actively participate by reading their glucose monitor and adjusting the delivery rate/dose based on information provided by the monitor. For a no-loop system (e.g. a patch-pump), the pump would deliver the glucagon formulation at a fixed (or basal) dose. The no-loop system can be used without a glucose monitor and without a glucose sensor if so desired.

In one aspect of the present invention there is disclosed a glucagon delivery apparatus comprising a reservoir containing a therapeutic composition comprising glucagon, a glucagon analogue, or a salt form of either thereof, a sensor configured to measure a patient's blood glucose level, and an electronic pump configured to intradermally, subcutaneously or intramuscularly deliver at least a portion of the therapeutic composition to a patient based on the patient's measured blood glucose level. The sensor can be positioned on the patient such that it contacts the patient's blood or contacts the patient's interstitial fluid or both. The sensor can be configured to transmit data (for example, wirelessly, via radio frequency, or via a wired connection) to a processor configured to control operation of the electronic pump. The processor can be configured to control operation of the pump based, at least in part, on the data obtained by the sensor. In one instance, the processor can be configured to control operation of the pump to intradermally, subcutaneously or intramuscularly inject at least a portion of the composition if the data obtained by the sensor indicates a glucose level below a defined threshold or indication that a defined threshold will breached in a particular period of time (e.g., an indication of impending hypoglycemia or an indication that the blood glucose levels will fall to below 70, 60, or 50 mg/dL within a certain period of time (e.g., within 30, 25, 20, 15, 10, 9, 8, 7, 6, 5, 4, 3, 2, or 1 minute(s)). Such an indication can be determined by identifying a downward trend of blood glucose levels (e.g., by the blood glucose monitoring device) as well as the speed or trajectory of this downward trend. The glucagon delivery apparatus can also include a monitor configured to communicate information indicative of the patient's glucose level. The monitor can include a speaker or a display device, or both. The monitor can be configured to communicate an alert when a glucose level of the patient is estimated to be at a defined threshold. Still further, the apparatus can be configured to allow manual adjustment of at least one of a delivery rate and a dose of the composition intradermally, subcutaneously or intramuscularly delivered by the pump.

In certain instances, the composition does not include a drug capable of decreasing the blood glucose level in the patient, nor are the compositions described used in combination in certain instances. Similarly, and in certain instances, the apparatus can be configured such that it is not capable of injecting a composition comprising a drug capable of decreasing the blood glucose level in the patient (e.g., the apparatus does not include such a composition in its reservoir to be administered to a patient). In other instances, the composition can also include a drug capable of decreasing the blood glucose level in the patient. Similarly, and in certain instances, the apparatus can be configured such that it is capable of injecting a composition comprising a drug capable of decreasing the blood glucose level (e.g., the apparatus includes in its reservoir a second composition having such a drug). The reservoir of the apparatus can have a single container or can have multiple containers for multiple compositions (e.g. each container may contain only a single composition). By way of example only, a reservoir having at least two containers can include one composition in one container that increases blood glucose levels (e.g., glucagon containing composition) and another that decreases blood glucose levels in a second container. This can result in the apparatus of the present invention operating as a fully operational artificial pancreas. Non-limiting examples of drugs that can decrease blood glucose levels in the patient include insulin, an insulin mimetic peptide, incretin, or an incretin mimetic peptide.

In certain aspects of the present invention, the apparatus is configured to be a closed-loop system. In other instances, it is configured to be an open-loop system. In still further instances, it is configured to be a no-loop system.

The flowable composition than can be included in the reservoir of the apparatus can be a single-phase solution comprising the glucagon, glucagon analogue, or a salt form of either thereof, dissolved in a non-aqueous solvent. In certain instances, the glucagon, glucagon analogue, or a salt form of either thereof, can be fully solubilized in an aqueous solvent system or a non-aqueous solvent system. In particular instances, the glucagon, glucagon analogue, or a salt form of either thereof, can be fully solubilized in an aprotic polar solvent system. Therapeutic molecules typically require an optimal or beneficial ionization profile in order to exhibit prolonged stability when solubilized in an aprotic polar solvent system (this is analogous to the pH of optimal stability and/or solubility for a peptide dissolved in an aqueous solution). An optimal or beneficial ionization profile of a therapeutic molecule may be obtained by direct dissolution of the therapeutic agent in an aprotic polar solvent system containing a specified concentration of at least one ionization stabilizing excipient. Non-limiting compositions for use with the present invention are stable formulations containing at least one therapeutic molecule solubilized in an aprotic polar solvent system. In certain aspects the therapeutic molecule does not need to be previously dried from a buffered aqueous solution prior to reconstitution in the aprotic polar solvent system. In certain non-limiting aspects a therapeutic agent is directly dissolved (e.g. a powder as received from a commercial manufacturer or supplier) along with an effective amount of an ionization stabilizing excipient (e.g. a mineral acid such as hydrochloric acid or sulfuric acid) for establishing an appropriate ionization of the therapeutic agent in the aprotic polar solvent system.

Non-limiting examples of stable solutions of a therapeutic agent(s) solubilized in non-aqueous aprotic polar solvents (e.g. DMSO), can be prepared by adding a specific predetermined amount (i.e. concentration) of a compound, or combination of compounds, that function as an ionization stabilizing excipient. The concentration can be determined by titration studies using the therapeutic agent and the ionization stabilizing excipient. Without wishing to be bound by theory, it is believed that the ionization stabilizing excipient can act as a proton source (e.g., a molecule that can donate a proton to the therapeutic molecule) in the aprotic polar solvent system that may protonate the ionogenic groups on the therapeutic molecule such that the therapeutic molecule possesses an ionization profile having an improved physical and chemical stability in the aprotic polar solvent system relative to a formulation prepared with an excess or insufficiency of the ionization stabilizing excipient.

Certain embodiments are directed to a formulation of a therapeutic agent comprising a therapeutic agent at a concentration of at least, at most, or about 0.1, 1, 10, 50, or 100 mg/mL to 150, 200, 300, 400, or 500 mg/mL or up to the solubility limit of the therapeutic agent in the aprotic polar solvent system comprising a concentration of at least one ionization stabilizing excipient that provides physical and chemical stability to the therapeutic agent. In certain aspects the therapeutic agent is a peptide. The formulation can comprise an ionization stabilizing excipient at a concentration of at least, at most, or about 0.01, 0.1, 0.5, 1, 10, or 50 mM to 10, 50, 75, 100, 500, 1000 mM, or up to the solubility limit of the ionization stabilizing excipient in the aprotic polar solvent system. In certain aspects the ionization stabilizing excipient concentration is between 0.1 mM to 100 mM. In certain embodiments the ionization stabilizing excipient may be a suitable mineral acid, such as hydrochloric acid or sulfuric acid. In certain aspects the ionization stabilizing excipient may be an organic acid, such as an amino acid, amino acid derivative, or the salt of an amino acid or amino acid derivative (examples include glycine, trimethylglycine (betaine), glycine hydrochloride, and trimethylglycine (betaine) hydrochloride). In a further aspect the amino acid can be glycine or the amino acid derivative trimethylglycine. In certain aspects a peptide is less than 150, 100, 75, 50, or 25 amino acids. In further aspects the aprotic solvent system comprises DMSO. The aprotic solvent can be deoxygenated, e.g., deoxygenated DMSO. In certain aspects the therapeutic agent is glucagon, a glucagon analogue, or salt thereof.

Compositions to be used in conjunction with the present invention can be made by:

(a) calculating or determining the appropriate ionization stabilizing excipient or proton concentration needed to achieve a stabilizing ionization profile of a target therapeutic agent (e.g., a peptide(s) or small molecule(s)) in an aprotic polar solvent system; (b) mixing at least one ionization stabilizing excipient with the aprotic polar solvent system to attain an appropriate ionization environment that provides the ionization profile determined in step (a); and (c) solubilizing the target therapeutic agent(s) in the aprotic solvent having an appropriate environment to physically and chemically stabilize the therapeutic agent. In certain aspects the dissolution of the therapeutic agent and the addition of the ionization stabilizing excipient to the aprotic polar solvent system can be done in any order or concurrently, thus the ionization stabilizing excipient can be mixed first followed by dissolution of the therapeutic agent, or the therapeutic agent can be dissolved followed by addition of the ionization stabilizing excipient to the solution, or the ionization stabilizing excipient and the therapeutic agent can be added or dissolved in an aprotic polar solvent system concurrently. In a further aspect the entire amount of a component (e.g., a therapeutic agent or an ionization stabilizing excipient) need not to be mixed at a particular point; that is, a portion of the one or more components can be mixed first, second, or concurrently, and another portion mixed at another time, first, second, or concurrently. In certain aspects the therapeutic agent can be a peptide, and the ionization stabilizing excipient may be a suitable mineral acid, such as hydrochloric acid or sulfuric acid. In certain aspects the peptide(s) is less than 150, 100, 75, 50, or 25 amino acids. The concentration of the therapeutic agent and/or ionization stabilizing excipient added to the solution can be between 0.01, 0.1, 1, 10, 100, 1000 mM to its solubility limit, including all values and ranges there between. In certain aspects the aprotic polar solvent system is deoxygenated. In a further aspect the aprotic polar solvent system comprises, consists essentially of, or consists of DMSO or deoxygenated DMSO.

In other non-limiting aspects a flowable composition can further comprise a carbohydrate, a sugar alcohol, a preservative, and optionally an acid. In one instance, the aprotic polar solvent can be DMSO, the carbohydrate can be trehalose, the sugar alcohol can be mannitol, the preservative can be metacresol, and the optional acid can be sulfuric acid. The composition can include at least 80 wt. % of the aprotic polar solvent, 3 to 7 wt. % of the carbohydrate, 1 to 5 wt. % of the sugar alcohol, 0 to 1 wt. %, and 0 wt. % to less than 1 wt. % of the acid. The composition can comprise, consists essentially of, or consist of glucagon, the glucagon analogue, or the salt form of either thereof, the aprotic polar solvent, the carbohydrate, the sugar alcohol, and optionally the acid. The composition can have an initial water content of 0 to less than 15 wt. %, 0 to less than 3 wt. %, 3 to 10 wt. %, or 5 to 8 wt. %. The glucagon, glucagon analogue, or salt form of either thereof, can have been previously dried from a buffered aqueous solution, wherein the dried glucagon, glucagon analogue, or salt form of either thereof, has a first ionization profile that corresponds to an optimal stability and solubility for the glucagon, glucagon analogue, or salt form thereof, wherein the dried glucagon, glucagon analogue, or salt form of either thereof, is reconstituted into an aprotic polar solvent and has a second ionization profile in the aprotic polar solvent, and wherein the first and second ionization profiles are within 1 pH unit of one another. The first or second or both ionization profiles can correspond to the ionization profile of glucagon when solubilized in an aqueous solution having a pH range of about 1 to 4 or 2.5 to 3.5.

In another instance, the flowable composition can be structured as a two-phase mixture of a powder dispersed in a liquid that is a non-solvent to the solid, where the powder comprises the glucagon, glucagon analogue, or a salt form of either thereof, and where the liquid is a pharmaceutically acceptable carrier, where the powder is homogeneously contained within a pharmaceutically acceptable carrier. The flowable composition can be a paste, slurry, or suspension. The powder can have a mean particle size ranging from 10 nanometers (0.01 microns) to about 100 microns, with no particles being larger than about 500 microns.

Due to the stability of the glucagon formulations being used with the apparatuses of the present invention, said formulations can be pre-loaded and stored in the reservoir and used over a period of time when exposed to room or body temperature (e.g., at least 1, 2, 3, 4, 5, 6, 7, 14, 21, 30, 45, or 60 days). This allows the apparatuses to be used as closed-loop, open-loop, or no-loop pump devices for maintaining appropriate blood glucose levels to prevent or treat hypoglycemia in the patient. In particular instances, the composition is capable of remaining stable and flowable after being stored for one month or 6 months or 12 months or 18 months at room temperature (e.g. 20-25° C.).

“Coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art.

Further, a device or system that is configured in a certain way is configured in at least that way, but it can also be configured in other ways than those specifically described.

A peptide's “optimal stability and solubility” refers to the pH environment wherein solubility of the peptide is high (at or near the maximum on a solubility versus pH profile, or suitable for the requirements of the product) and its degradation minimized relative to other pH environments. Notably, a peptide may have more than one pH of optimal stability and solubility. This can also refer to the ionization profile (e.g. protonation state) that a peptide possesses when solubilized in an aqueous solution having a pH of optimal stability for that peptide. A person having ordinary skill in the art can easily ascertain a given peptide's optimal stability and solubility by referencing literature or by performing assays.

The term “dissolution” as used herein refers to a process by which a material(s) in a gas, solid, or liquid state becomes a solute(s), a dissolved component(s), of a solvent, forming a solution of the gas, liquid, or solid in the solvent. In certain aspects a therapeutic agent or an excipient, e.g., an ionization stabilizing excipient, is present in an amount up to its solubility limited or is fully solubilized. The term “dissolve” refers to a gas, liquid, or solid becoming incorporated into a solvent to form a solution.

The term “excipient” as used herein refers to a natural or synthetic substance formulated alongside the active or therapeutic ingredient (i.e. an excipient is an ingredient that is not the active ingredient) of a medication, included for the purpose of stabilization, bulking, or to confer a therapeutic enhancement on the active ingredient in the final dosage form, such as facilitating drug absorption, reducing viscosity, enhancing solubility, adjusting tonicity, mitigating injection site discomfort, depressing the freezing point, or enhancing stability. Excipients can also be useful in the manufacturing process, to aid in the handling of the active substance concerned such as by facilitating powder flowability or imparting non-stick properties, in addition to improving in vitro stability such as prevention of denaturation or aggregation over the expected shelf life.

As used herein an “ionization stabilizing excipient” is an excipient that establishes and/or maintains a particular ionization state for a therapeutic agent. In certain aspects the ionization stabilizing excipient can be, or includes, a molecule that donates at least one proton under appropriate conditions or is a proton source. According to the Bronsted-Lowry definition, an acid is a molecule that can donate a proton to another molecule, which by accepting the donated proton may thus be classified as a base. As used in this application, and as will be understood by the skilled technician, the term “proton” refers to the hydrogen ion, hydrogen cation, or H⁺. The hydrogen ion has no electrons and is composed of a nucleus that typically consists solely of a proton. Specifically, a molecule that can donate a proton to the therapeutic agent is considered an acid or proton source, regardless of whether it is completely ionized, mostly ionized, partially ionized, mostly unionized, or completely unionized in the aprotic polar solvent.

As used herein a “mineral acid” is an acid that is derived from one or more inorganic compounds. Accordingly, mineral acids may also be referred to as “inorganic acids.” Mineral acids may be monoprotic or polyprotic (e.g. diprotic, triprotic, etc.). Examples of mineral acids include hydrochloric acid (HCl), sulfuric acid (H₂SO₄), and phosphoric acid (H₃PO₄).

As used herein an “organic acid” is an organic compound with acidic properties (i.e. can function as a proton source). Carboxylic acids are one example of organic acids. Other known examples of organic acids include, but are not limited to, alcohols, thiols, enols, phenols, and sulfonic acids. Organic acids may be monoprotic or polyprotic (e.g. diprotic, triprotic, etc.)

“Charge profile,” “charge state,” “protonation state,” “ionization state,” and “ionization profile” may be used interchangeably and refer to the ionization state (i.e. due to protonation and/or deprotonation) of the peptide's ionogenic groups.

“Therapeutic agent” encompasses peptide compounds together with pharmaceutically acceptable salts thereof. Useful salts are known to those skilled in the art and include salts with inorganic acids, organic acids, inorganic bases, or organic bases. Therapeutic agents useful in the present invention are those peptide compounds that affects a desired, beneficial, and often pharmacological, effect upon administration to a human or an animal, whether alone or in combination with other pharmaceutical excipients or inert ingredients.

“Peptide,” “polypeptide” and “peptide compound” refer to polymers of up to about 100 or more preferably up to about 80 amino acid residues bound together by amide (CONH) linkages. Analogs, derivatives, agonists, antagonists and pharmaceutically acceptable salts of any of the peptide compounds disclosed here are included in these terms, and the amino acids residues that comprise the peptide can be proteinogenic and/or non-proteinogenic. The terms also include peptides and peptide compounds that have D-amino acids, modified, derivatized or naturally occurring amino acids in the D- or L-configuration and/or peptomimetic units as part of their structure.

The term “glucagon” refers to the glucagon peptide, analogues thereof, and salt forms of either thereof. The glucagon peptide, analogues thereof, and salt forms may be derived from synthetic or recombinant processes.

As used herein, a “co-formulation” is a formulation that contains two or more therapeutic agents dissolved in an aprotic polar solvent system. The therapeutic agents may belong to the same class (for example, a co-formulation comprising two or more therapeutic peptides, such as insulin and pramlintide), or the therapeutic agents may belong to different classes (for example a co-formulation comprising one or more therapeutic small molecules and one or more therapeutic peptide molecules, such as GLP-1 and lisofylline).

“Patient,” “subject,” or “individual” refers to a mammal (e.g., human, primate, dog, cat, bovine, ovine, porcine, equine, mouse, rate, hamster, rabbit, or guinea pig). In particular aspects, the patient is a human. In preferred aspects of the present invention, the patient has congenital hyperinsulinism (CHI) and/or is less than 20, 19, 18, 17, 16, 15, 14, 13, 12, 11, 10, 9, 8, 7, 6, 5, 4, 3, 2, or less than 1 year(s) old. In certain aspects, the patient is an infant less than 1 year old or less than 9 months old or less than 6 months old or less than 3 months old.

“Inhibiting” or “reducing” or any variation of these terms includes any measurable decrease or complete inhibition to achieve a desired result.

“Effective” or “treating” or “preventing” or any variation of these terms means adequate to accomplish a desired, expected, or intended result.

As used herein, the term “aprotic polar solvent” refers to a polar solvent which does not contain acidic hydrogen and thus does not act as a hydrogen bond donor. Polar aprotic solvents include, but are not limited to dimethylsulfoxide (DMSO), dimethylformamide (DMF), ethyl acetate, n-methyl pyrrolidone (NMP), dimethylacetamide (DMA), and propylene carbonate. An “aprotic polar solvent system” refers to a solution wherein the solvent is a single aprotic polar solvent (for example, neat DMSO), or a mixture of two or more aprotic polar solvents (for example, a mixture of DMSO and NMP).

“Single-phase solution” refers to a solution prepared from a powder dissolved in a solvent, or solvent system (e.g., mixture of two or more solvents), wherein the particulate matter is completely dissolved in the solvent and there is no longer particulate matter visible, such that the solution can be described as optically clear. A single-phase solution may be colorless or colored (e.g. light yellow discoloration).

“Buffer” refers to a weak acid or base that prevents rapid or significant changes in the pH of a solution following the addition of other acids and/or bases. When buffering agent are added to water, a buffered solution is formed. For example, a buffer solution may contain both a weak acid and its conjugate base, or a weak base and its conjugate acid. In common chemical usage, a pH buffer is a substance or a mixture of substances, which permits solutions to resist large changes in pH upon addition of small amounts of H⁺ and OH⁻ ions. A common buffer mixture contains two substances, a conjugate acid (proton donor) and a conjugate base (proton acceptor). Together, the two species (the conjugate acid-base pair of a conjugate acid and conjugate base) resist large changes in pH of the solution by partially absorbing additions of H⁺ and OH⁻ ions to the solution.

“Non-volatile buffer” refers to a buffer where the buffer components are not sufficiently volatile that they may be removed from the composition during drying (e.g., during lyophilization). Glycine, citrate, or phosphate buffers, or mixtures thereof are a few non-limiting examples of non-volatile buffers. In preferred instances, glycine buffers can be used as the non-volatile buffer.

“Isoelectric point” (pI) of a peptide corresponds to the pH value where the overall net charge of the peptide is zero. Due to their varying composition with respect to their primary structures, peptides may have varying isoelectric points. In peptides there may be many charged groups (e.g., ionogenic groups that have been protonated or deprotonated) and at the isoelectric point the net sum of all these charges is zero, i.e. the number of negative charges balances the number of positive charges. At a pH above the isoelectric point the overall net charge of the peptide will be negative, and at pH values below the isoelectric point the overall net charge of the peptide will be positive. There are multiple methods known in the art for determining the isoelectric point of a peptide, including experimental methods such as isoelectric focusing, and theoretical methods where the isoelectric point may be estimated from the amino acid sequence of the peptide by computational algorithms.

“Reconstituted,” when referring to a pharmaceutical composition, refers to a composition which has been formed by the addition of an appropriate non-aqueous solvent to a solid material comprising the active pharmaceutical ingredient. Pharmaceutical compositions for reconstitution are typically applied where a liquid composition with acceptable shelf-life cannot be produced. An example of a reconstituted pharmaceutical composition is the solution which results when adding a biocompatible aprotic polar solvent (e.g., DMSO) to a freeze dried composition.

“Primary structure” refers to the linear sequence of amino acid residues that comprise a peptide/polypeptide chain.

“Analogue” and “analog,” when referring to a peptide, refers to a modified peptide wherein one or more amino acid residues of the peptide have been substituted by other amino acid residues, or wherein one or more amino acid residues have been deleted from the peptide, or wherein one or more amino acid residues have been added to the peptide, or any combination of such modifications. Such addition, deletion or substitution of amino acid residues can take place at any point, or multiple points, along the primary structure comprising the peptide, including at the N-terminal of the peptide and/or at the C-terminal of the peptide. Naturally occurring proteinogenic amino acids may be substituted with other proteinogenic amino acids, or non-proteinogenic amino acids. One example of a glucagon analogue comprising both proteinogenic and non-proteinogenic amino acid residues is dasiglucagon (Zealand Pharma A/S).

“Derivative,” in relation to a parent peptide, refers to a chemically modified parent peptide or an analogue thereof, wherein at least one substituent is not present in the parent peptide or an analogue thereof. One such non-limiting example is a parent peptide which has been covalently modified. Typical modifications are amides, carbohydrates, alkyl groups, acyl groups, esters, pegylations and the like.

An “amphoteric species” is a molecule or ion that can react as an acid as well as a base. These species can either donate or accept a proton. Examples include amino acids, which possess both amine and carboxylic acid functional groups. Amphoteric species further include amphiprotic molecules, which contain at least one hydrogen atom, and have the ability to donate or accept a proton.

“Insulin secretion inhibiting drugs” refers to compounds such as diazoxide, octreotide, or calcium channel blockers that can inhibit insulin secretion.

“Non-aqueous solvent” refers to a solvent or solvent system that contains either no or minimal moisture content.

A “therapeutically equivalent” drug is one that has essentially the same effect in the treatment of a disease or condition as one or more other drugs. A drug that is therapeutically equivalent may or may not be chemically equivalent, bioequivalent, or generically equivalent.

“Water” or “moisture” content of the formulations of the present invention refers to the total amount of water present in a given formulation. In general, there are two types of moisture in a formulation and include (1) the initial moisture content of the formulation and (2) the total moisture content of the formulation. The initial moisture content and the total moisture content of a formulation are equal immediately after the formulation is prepared. However, during storage moisture may enter the formulation such that the total moisture content will increase above the initial moisture content. By way of example, the formulations of the present invention can be hygroscopic in that the formulation after initially being prepared may have 1 wt. % water content but after a period of time (e.g., storage for one month), its water content increases to 2 wt. %. Therefore, the total moisture or total water content in such a formulation would be 2 wt. %, above the 1 wt. % initial moisture content of the formulation. The initial moisture content of a formulation can be contributed by multiple sources. For example, water may be added as a co-solvent (for example, to depress the freezing point of the formulation), and/or residual moisture may be present in the powder following drying (e.g. via lyophilization) of the initial aqueous solution containing the peptide. The amount of residual moisture remaining due to incomplete removal during drying varies according to, among other factors, the instrument, batch size, processing parameters, but is typically less than 10 wt. %.

Alternatively, water may be used as a co-solvent in the context of the present formulations, where the water can be used to depress the freezing point of the formulation. For example, a formulation could include 10 wt. % water as a co-solvent such that the formulation after its initial preparation has 10 wt. % water but after a period of time (e.g., storage for one month), its water content increases above 10 wt % (e.g., 11 wt. %). In this example, the initial moisture or initial water content in such a formulation is 10 wt. %, but the total water or moisture content is 11 wt. %. The formulations of the present invention can have an initial water or initial moisture content of less than 15 wt. %, less than 10 wt. %, less than 5 wt. %, less than 4 wt. %, less than 3 wt. %, less than 2 wt. %, or less than 1 wt. % after the formulations have been prepared. In specific embodiments, the composition can have an initial water content of 0 to less than 15 wt. %, 0 to less than 3 wt. %, 3 to 10 wt. %, or 3 to 5 wt. %.

“Bioavailability” refers to the extent to which the therapeutic agent, such as a peptic compound, is absorbed from the formulation.

“Systemic,” with respect to delivery or administration of a therapeutic agent, such as a peptide compound, to a subject, that therapeutic agent is detectable at a biologically significant level in the blood plasma of the subject.

“Controlled release” refers to the release of the therapeutic agent at such a rate that blood (e.g., plasma) concentrations are maintained within the therapeutic range, but below toxic concentrations over a period of time of about one hour or longer, preferably 12 hours or longer.

“Pharmaceutically acceptable carrier” refers to a pharmaceutically acceptable solvent, suspending agent or vehicle for delivering a drug compound of the present invention to a mammal such as an animal or human.

“Pharmaceutically acceptable” ingredient, excipient or component is one that is suitable for use with humans and/or animals without undue adverse side effects (such as toxicity, irritation and allergic response) commensurate with a reasonable benefit/risk ratio.

“Chemical stability,” when referring to a therapeutic agent, such as a peptide or salt thereof, refers to an acceptable percentage of degradation products produced by chemical pathways such as oxidation or hydrolysis is formed. In particular, a formulation is considered chemically stable if no more than about 30% degradation products are formed after one year of storage at the intended storage temperature of the product (e.g., room temperature); or storage of the product at 30° C./65% relative humidity for one year; or storage of the product at 40° C./75% relative humidity for one month, and preferably three months. In some embodiments, a chemically stable formulation has less than 20%, less than 15%, less than 10%, less than 5%, less than 4%, less than 3%, less than 2%, or less than 1% degradation products formed after an extended period of storage at the intended storage temperature of the product.

“Physical stability,” when referring to a therapeutic agent, such as a peptide or salt thereof, refers to an acceptable percentage of aggregates (e.g., soluble aggregates such as dimers, trimers and larger forms) being formed. In particular, a formulation is considered physically stable if no more that about 15% aggregates are formed after one year of storage at the intended storage temperature of the product (e.g., room temperature); or storage of the product at 30° C./65% relative humidity for one year; or storage of the product at 40° C./75% relative humidity for one month, preferably two months, and most preferably three months. In some embodiments, a physically stable formulation has less than less than 15%, less than 10%, less than 5%, less than 4%, less than 3%, less than 2%, or less than 1% aggregates formed after an extended period of storage at the intended storage temperature of the product.

“Stable formulation” refers to at least about 65% chemically and physically stable therapeutic agents, such as peptides or salts thereof, remain after two months of storage at room temperature. Particularly preferred formulations are those in which at least about 80%, 85%, 90%, 91%, 92%, 93%, 94%, 95%, 96%, 97%, 98%, or 99% chemically and physically stable therapeutic agent remains under these storage conditions. Especially preferred stable formulations are those which do not exhibit degradation after sterilizing irradiation (e.g., gamma, beta or electron beam).

“Mammal” or “mammalian” includes murine (e.g., rats, mice) mammals, rabbits, cats, dogs, pigs, and primates (e.g., monkey, apes, humans). In particular aspects in the context of the present invention, the mammal can be murine or human. The patient can be a mammal or a mammalian patient.

“Parenteral injection” refers to the administration of therapeutic agents, such as peptide compounds, via injection under or through one or more layers of skin or mucus membranes of an animal, such as a human. Standard parenteral injections are given into the intradermal, subcutaneous, or intramuscular region of an animal, e.g., a human patient. In some embodiments, a deep location is targeted for injection of a therapeutic agent as described herein.

The term “about” or “approximately” or “substantially unchanged” are defined as being close to as understood by one of ordinary skill in the art, and in one non-limiting embodiment the terms are defined to be within 10%, preferably within 5%, more preferably within 1%, and most preferably within 0.5%. Further, “substantially non-aqueous” refers to less than 5%, 4%, 3%, 2%, 1%, or less by weight or volume of water.

The use of the word “a” or “an” when used in conjunction with the term “comprising” in the claims and/or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The apparatuses, compositions, and methods of the present invention can “comprise,” “consist essentially of,” or “consist of” any of the claimed elements or steps disclosed throughout the specification. With respect to the transitional phase “consisting essentially of,” in one non-limiting aspect, a basic and novel characteristic of the apparatuses of the present invention are their ability to deliver stable glucagon formulations to patients via closed-loop, open-loop, or no-loop pump-based devices.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1 is a perspective view of a first embodiment of the present glucagon delivery apparatuses.

FIG. 2 is a cross-sectional side view of various components of the glucagon delivery apparatus of FIG. 1 shown coupled to a patient.

FIG. 3 is a schematic depicting various components of the glucagon delivery apparatus of FIG. 1.

FIGS. 4A-4C are side views of reservoirs containing various compositions of the present disclosure that are suitable for use in some embodiments of the present glucagon delivery apparatuses.

FIG. 4D is a top view of a reservoir suitable for use in some embodiments of the present glucagon delivery apparatuses.

FIG. 5 depicts an illustrative flow chart of one example of closed-loop control of one embodiment of the present glucagon delivery apparatuses.

FIG. 6 provides data that indicates a clinically significant reduction in the amount of glucose that must be infused (i.e. the glucose infusion rate (GIR)) to maintain the patient's glucose levels in the euglycemic range when used with and without glucagon CSI.

### DETAILED DESCRIPTION OF ILLUSTRATIVE EMBODIMENTS

Prior to the present invention, the typical process of treating CH includes diazoxide or octreotide to block insulin release from the pancreas, but these drugs have significant side effects and are effective in less than half of all cases. The other CH therapy is continuous infusion of an aqueous solution of dextrose (for example, a 50% (w/v) dextrose solution referred to hereafter as D50). However, D50 therapy typically requires high glucose infusion rate (GIR) via a peripherally inserted central catheter, or PICC line, which must be implanted surgically. The PICC line is a source of infection for the patient and a high GIR can cause fluid overload, which can lead to heart failure, pulmonary edema, and cyanosis.

The present invention offers a solution to the current D50 treatment method for CH. The solution is premised, in part, on a discovery that a stable and flowable glucagon formulation delivered as a continuous subcutaneous infusion (CSI) can be administered before or during D50 treatment, which can then result in lowering the GIR of D50 over a shorter time period than without the use of glucagon CSI. In one non-limiting embodiment, use of a glucagon formulation of the present invention in combination with a patch-pump (e.g. OmniPod) can enable treatment via a more convenient subcutaneous administration rather than the current paradigm which requires a surgically implanted PICC line, followed by years of IV infusion of D50. Without wishing to be bound by theory, it is believed that glucagon CSI can result in lower level D50 administration or even complete removal/avoidance of D50 administration altogether.

These and other aspects of the present invention are provided in non-limiting detail in the following subsections.

### A. Glucagon Delivery Apparatuses and Related Methods

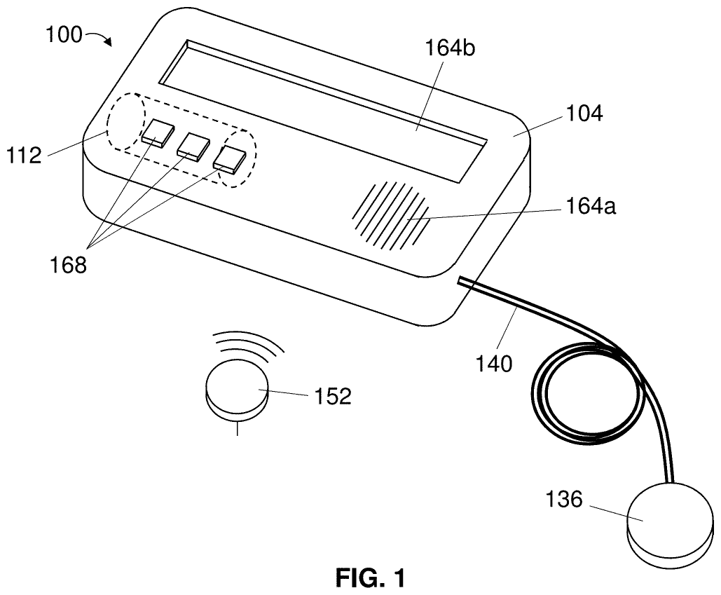

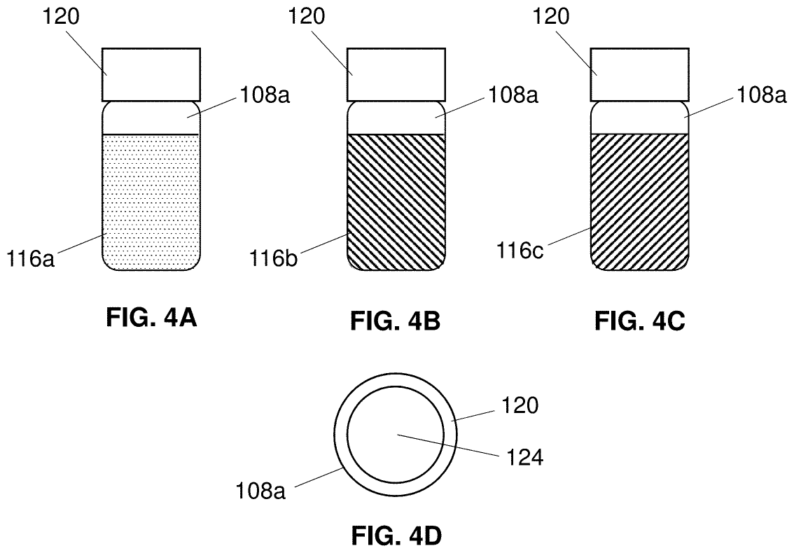

Referring now to FIGS. 1-4, shown therein and designated by the reference numeral 100 is a first non-limiting embodiment of the present glucagon delivery apparatuses. In the depicted embodiment, apparatus 100 comprises a housing 104, which generally functions to locate and/or secure components of apparatus 100 relative to one another. In the embodiment shown, glucagon delivery apparatus 100 is configured to intradermally, subcutaneously or intramuscularly deliver a composition comprising glucagon to a patient.

In the depicted embodiment, apparatus 100 comprises a reservoir 108a, which in this embodiment, may be disposed and/or disposable within housing 104. For example, in this embodiment, housing 104 defines and/or is configured to allow access to a receptacle 112, which may be dimensioned to receive and/or allow removal and/or replacement of reservoir 108a within housing 104.

In this embodiment, reservoir 108a may comprise a composition (e.g., 116a, 116b, 116c, and/or the like) (sometimes referred to collectively as “composition 116” or “compositions 116”). The present glucagon delivery apparatuses can be used with any suitable storage stable composition, such as, for example, the glucagon containing formulations described throughout the present application.

In the embodiment shown, reservoir 108a comprises a cap 120. In this embodiment, cap 120 includes a puncturable seal 124 (e.g., which may be punctured by a needle or other sharp object external to or within apparatus 100, for example, when reservoir 108a is inserted into receptacle 112, to allow for communication of composition 116 from reservoir 108a to pump 128). In this way, compositions 116 can be stored prior to use, which may be facilitated by the stability of the compositions.

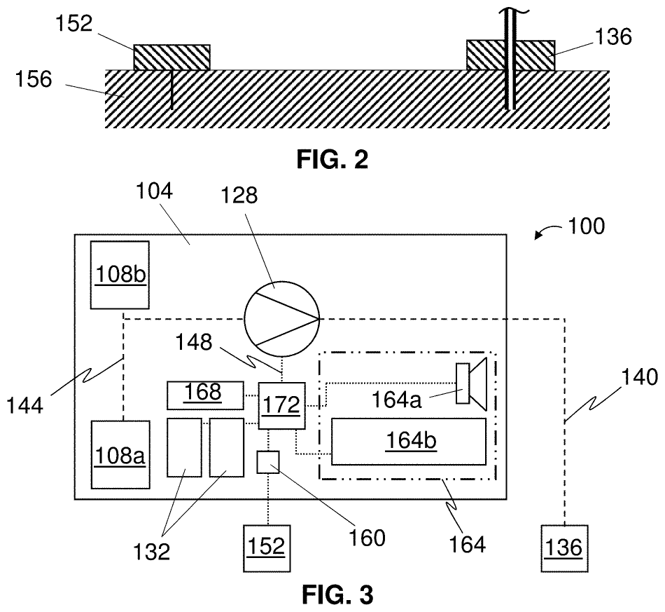

While some embodiments of the present glucagon delivery apparatuses do not comprise a composition having a protein or peptide capable of decreasing the blood glucose level of a patient, other embodiments may comprise a composition including a glucose-reducing formulation (e.g., insulin, an insulin memetic peptide, incretin, an incretin mimetic peptide, and/or the like, as described above). For example, some embodiments may comprise a (e.g., additional to reservoir 108a) reservoir 108b containing the glucose-reducing formulation, and, in such embodiments, pump 128 (described in more detail below) may be configured to intracutaneously delivery at least a portion of the glucose-reducing formulation (an example of such a configuration is depicted in FIG. 3, which may include a valve to selectively place either reservoir 108a or reservoir 108b in communication with pump 128). In these and similar embodiments, housing 104 may comprise a receptacle (e.g., 112) dimensioned to receive and/or allow removal and/or replacement of reservoir 108b within housing 104.

In the embodiment shown, apparatus 100 comprises an electronic pump 128 configured to intracutaneously delivery at least a portion of the composition to a patient. Pumps of the present disclosure can comprise any suitable pump, such as, for example, positive displacement pumps (e.g., gear pumps, screw pumps, peristaltic pumps, piston pumps, plunger pumps, and/or the like), centrifugal pumps, and/or the like. In this embodiment, pump 128 is electronic (e.g., is configured to be actuated electrically, for example, by an electric motor with power supplied from a battery 132); however, in other embodiments, the pump may be actuated manually (e.g., via application of force by a user, for example, to a plunger, lever, crank, and/or the like). In this embodiment, pump 128 is in communication with a needle 136 via an (e.g., flexible) conduit 140 such that actuation of pump 128 may cause communication of composition 116 from reservoir 108a, through conduit 140, and into the patient via needle 136 (e.g., which, in some embodiments, may be configured to be received within an implanted port of the patient). An example of such composition communication is depicted in FIG. 3, in which composition communication is indicated by dashed lines 144, and electrical communication is indicated by dotted lines 148.

In the embodiment shown, apparatus 100 comprises a sensor 152 configured to obtain data indicative of a glucose level within interstitial fluid of the patient (e.g., by measuring a current generated as glucose oxidase (GOx) catalyzes the reaction of glucose in the interstitial fluid with oxygen). The level can then be used to determine the blood glucose level of the patient or can be used to determine how much of the glucagon formulation to administer to the patient. For example, and referring particularly to FIG. 2, in this embodiment, a portion of sensor 152 (e.g., which may include a needle, electrodes, and/or the like) is inserted into a patient's skin 156 and is in communication with the interstitial fluid.

In the embodiment shown, sensor 152 is configured to transmit data wirelessly. For example, in this embodiment, sensor 152 is configured to transmit data via radio frequency (e.g., whether in response to a signal generated by a reader 160 and/or facilitated by a battery in electrical communication with the sensor). However, in other embodiments, sensor 152 can be configured to transmit data via a wired connection.

In the embodiment shown, apparatus 100 comprises a monitor 164 configured to communicate information indicative of the glucose level within the interstitial fluid of the patient. Monitors 164 of the present disclosure can comprise any suitable monitor, and can be configured to communicate information audibly (e.g., via a speaker 164a), tactilely (e.g., via a vibratory motor), visually (e.g., via a display device 164b), and/or the like. For example, in this embodiment, monitor 164 comprises a speaker 164a and a display device 164b. While monitor 164 is depicted as attached to housing 104 of apparatus 100, in other embodiments, monitors (or components thereof, such as, for example, speaker 164a or display device 164b) may be physically separate from housing 104 (e.g., and in wireless and/or wired communication with other components of apparatus 100). In this way, by receiving information communicated by monitors 164, a patient using apparatus 100 may gain insight into how food intake, physical activity, medication, illness, and/or the like impact blood glucose levels.

In the embodiment shown, monitor 164 can be configured to communicate alerts under any suitable circumstance (e.g., triggers for which may be stored within a memory in electrical communication with processor 172). To illustrate, in this embodiment, apparatus 100 is configured such that monitor 164 communicates an alert when a glucose level within interstitial fluid of the patient is estimated to be at least one of: above a threshold (e.g., indicating an existing or impending hypoglycemic condition) and below a threshold (e.g., indicating an existing or impending hyperglycemic condition). Processor 172 may detect impending conditions by analyzing data received from sensor 152 over a time period to anticipate a patient's blood glucose level at a future time period (e.g., by determining trends within the patient's blood glucose level over time).

In this embodiment, apparatus 100 is configured to allow manual adjustment of at least one of a delivery rate and a dose of the composition intracutaneously delivered by pump 128. For example, in the embodiment shown, apparatus 100 comprises one or more user input devices (e.g., buttons) 168. User input devices 168 can be configured to allow a user to activate and/or deactivate apparatus 100 and/or pump 128, set a time and/or time period for activation and/or deactivation of apparatus 100 and/or pump 128, set a desired blood glucose level, set a desired composition delivery rate and/or dose (e.g., basal and/or bolus doses), and/or the like. User input devices 168 may work in conjunction with monitor 164 (or a display device 164b thereof) (e.g., to provide information to assist a user in interacting with apparatus 100, to provide for menu navigation, to display current parameters (e.g., target blood glucose level, composition delivery rate and/or dose, and/or the like), and/or the like). While in the depicted embodiment, user input devices 168 comprise buttons, in other embodiments, user input devices 168 can comprise any suitable structure, such as, for example, touch sensitive surface(s) of a display device 164b.

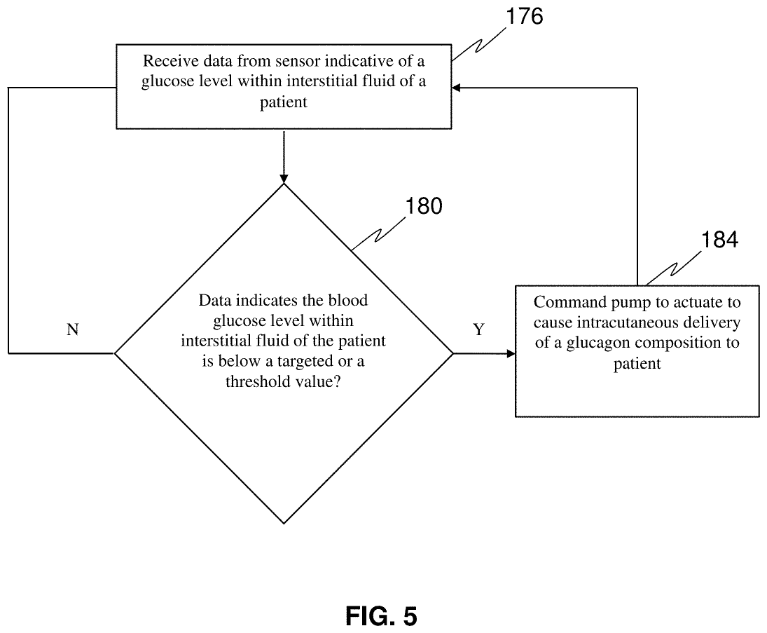

In the embodiment shown, apparatus 100 comprises a processor 172 configured to control operation of pump 128. In the embodiment shown, processor 172 control can be open-loop or closed-loop (e.g., based, at least in part, on data obtained by sensor 152). To illustrate, in this embodiment, processor 172 is configured to control operation of pump 128 to intracutaneously inject at least a portion of composition 116 if the data obtained by the sensor indicates a blood glucose level within interstitial fluid of the patient below a threshold (e.g., indicating an existing or impending hyperglycemic condition). FIG. 5 provides an illustrative flow chart of such closed-loop processor-based control. For example, at step 176, processor may receive data from sensor 152 indicative of the glucose level within interstitial fluid of the patient (e.g., through communication with reader 160). At step 180, in this embodiment, processor 172 may compare the received data to a targeted or threshold value. In the depicted embodiment, at step 184, if the data indicates a blood glucose level within interstitial fluid of the patient is below the targeted or threshold value, processor 172 may command pump 128 to actuate to cause intracutaneous delivery of composition 116 to the patient. Embodiments configured for such closed-loop control may require no input from a patient, and may be suited for treating patients having, for example, type II insulin dependent diabetes, post-bariatric surgery reactive hypoglycemia, hypoglycemia associated autonomic failure, insulinoma, and/or the like.

In some embodiments (e.g., 100), the present apparatuses can be configured to communicate (e.g., via a display 164b) data indicative of current blood glucose level to a patient, whereby the patient may adjust the delivery rate, dose, and/or the like of composition 116 (e.g., controlling apparatus 100 in an open-loop fashion). Embodiments configured for such open-loop control may be suited for treating patients having, for example, type I insulin dependent diabetes, type II insulin dependent diabetes, and/or the like.

Some embodiments may be configured to provide intradermal, subcutaneous or intramuscular delivery of composition 116 in a no-loop fashion. For example, some embodiments may be configured such that pump 128 actuates to deliver a fixed (e.g., basal) dose of composition 116. In these and similar embodiments, sensor 152, reader 160, monitor 164, user input devices 168, processor 172, and/or the like may be omitted. Such embodiments may be suitable for treating patients having, for example, congenital hyperinsulinism, post-bariatric surgery reactive hypoglycemia, and/or the like.

Some embodiments of the present methods for treating CH in a patient comprise using a glucagon delivery apparatus (e.g., 100) to intradermally, subcutaneously or intramuscularly deliver at least a portion of a composition (e.g., 116) to the patient. In some embodiments, the patient has been diagnosed as having a blood glucose level from 0 mg/dL to less than 50 mg/dL or has an indication of impending hypoglycemia before delivery of the composition, and the patient has a blood glucose level from 50 mg/dL to 180 mg/dL within 1 to 30 minutes after delivery of the composition. In some embodiments, the patient has been diagnosed as having a blood glucose level between from 10 mg/dL to less than 40 mg/dL. In some embodiments, the patient has a blood glucose level from 50 mg/dL to 180 mg/dL within 1 to 30 minutes after delivery of the composition. In some embodiments, the patient has a blood glucose level from 50 mg/dL to 180 mg/dL within 1 to 15 minutes after delivery of the composition. In some embodiments, the patient has been diagnosed with type I, type II, or gestational diabetes. Some embodiments comprise measuring, with a sensor (e.g., 152), the blood glucose level of the patient.

### B. Commercially Available Glucagon Formulations

In addition to the glucagon formulations discussed above, it is also contemplated in the context of the present invention that commercially available glucagon formulations can be used in the context of the present invention for treating CH and ultimately reducing D50 GIR levels or even avoiding the need for D50 therapy. Non-limiting examples of commercially available glucagon formulations include the Glucagon Emergency Kit (Eli Lilly) and the GlucaGen rescue kit (Novo Nordisk), both of which are sold as powders that must be reconstituted with a diluent syringe at the time of administration and are prone to fibrillation and gelation during storage.

Determination of an effective amount or dose is well within the capability of those skilled in the art, especially in light of the detailed disclosure provided herein. Generally, the formulations to deliver these doses may contain a glucagon peptide present at a concentration from about 0.1 mg/mL up to the solubility limit of the peptide in the formulation to produce a solution, wherein the glucagon peptide is fully or completely solubilized in the aprotic polar solvent. This concentration is preferably from about 1 mg/mL to about 100 mg/mL, e.g., about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL.

### C. Treating Congenital Hyperinsulinism

Congenital hyperinsulinism (CH) is a genetic disorder of pancreatic (3-cell function characterized by failure to suppress insulin secretion in the presence of hypoglycemia, resulting in brain damage or death if inadequately treated. CHI is a relatively rare disease. For instance, it can affect 1 in 25,000 to 50,000 babies/infants. Germline mutations in several genes have been associated with congenital hyperinsulinism. These mutations can include, for example, the sulfonylurea receptor (SUR-1, encoded by ABCC8), an inward rectifying potassium channel (Kir6.2, encoded by KCNJ1 1), glucokinase (GCK), glutamate dehydrogenase (GLUD-1), short-chain L-3-hydroxyacyl-CoA (SCHAD, encoded by HADSC) and/or mitochondrial uncoupling protein 2 (UCP2). A non-limiting example of the application of the disclosed invention can include identifying a CHI patient that requires a glucose infusion rate (GIR) of 20 mg/(kg*min) to maintain a targeted euglycemic blood glucose level of 100 mg/dL. The patch-pump containing 5 mg/mL non-aqueous glucagon formulation can be turned on, delivering a continuous subcutaneous glucagon infusion rate of 5 mcg/(kg*hr). The glucose of the CHI patient will begin to rise, which will require the clinician to decrease the glucagon infusion rate to maintain the targeted 100 mg/dL blood glucose level. The infusion rate of the stable and soluble exogenous glucagon formulation can be increased (e.g. up to 25 mcg/(kg*hr)) to allow the GIR to drop sufficiently low (e.g. <8 mg/(kg*min)) such that the peripherally inserted central catheter may be removed from the patient.

### EXAMPLES

Some embodiments of the present disclosure will be described in greater detail by way of specific examples. The following examples are offered for illustrative purposes, and are not intended to limit any present invention in any manner. For example, those of skill in the art will readily recognize a variety of noncritical parameters which can be changed or modified to yield essentially the same results.

### Example 1

### Ionization Stabilized Glucagon Composition

As a non-limiting example of a stable and flowable glucagon formulation that may be used in the context of the present invention, the preparation of a stable, non-aqueous glucagon solution is described. In this example, glucagon solutions were prepared by dissolving glucagon peptide powder (Bachem AG) in acidified DMSO containing dissolved 5% (w/v) trehalose (from dihydrate) and optionally mannitol (2.9% (w/v)). The DMSO solution was acidified with 3.0-3.2 mM H₂SO₄ (from a 1.0 N sulfuric acid stock solution). Samples were stored in both glass and CZ (Crystal Zenith) vials (0.5-mL fill volume in 2-mL vials) and placed on stability 40° C./75% RH. Chemical stability of the samples was examined following 60 days using a glucagon stability indicating UHPLC-MS method.

The reversed-phase ultra-high-performance liquid chromatography-mass spectrometry (RP-UHPLC-MS) method used to assess chemical stability was a gradient method with mobile phases A and B respectively consisting of 1% (v/v) FA (Formic Acid) in water and 1% (v/v) FA in acetonitrile. A C8 column (2.1 mm I.D.×100 mm length, 1.7 micron particle size) was used with a column temperature of 60° C., a 0.55 mL/min flow rate, 5-μL sample injection volume and 280-nm detection wavelength. The chemical stability data provided in Table 1 indicate that the soluble non-aqueous glucagon formulation exhibits long-term stability at accelerated conditions in both glass and COP (CZ) container-closure systems.

TABLE 1
Chemical stability (provided as glucagon peak purity) for a
soluble, non-aqueous 5 mg/mL glucagon formulation following
60 days at 40° C./75% RH. Data is provided as the average
(±standard deviation) for N = 3 sample replicates.

| | | Physical | Glucagon Peak |
|---|---|---|---|
| CCS | Excipients | Appearance | Purity |
| Glass | 5% (w/v) Trehalose | Clear, Colorless | 90.2 (±0.1)% |
| | 3.0 mM H₂SO₄ | Solution | |
| CZ | 5% (w/v) Trehalose | Clear, Colorless | 87.5 (±0.4)% |
| | 3.0 mM H₂SO₄ | Solution | |
| Glass | 5% (w/v) Trehalose | Clear, Colorless | 90.1 (±0.3)% |
| | 2.9% (w/v) Mannitol | Solution | |
| | 3.2 mM H₂SO₄ | | |
| CZ | 5% (w/v) Trehalose | Clear, Colorless | 88.0 (±0.5)% |
| | 2.9% (w/v) Mannitol | Solution | |
| | 3.2 mM H₂SO₄ | | |

### Example 2

### Clinical Study Data On Effects of Glucagon CSI vis-à-vis Glucose D50 Treatment

An ongoing clinical trial is being performed to evaluate whether CSI-Glucagon (non-aqueous glucagon formulation in DMSO) can reduce or eliminate the glucose infusion requirement (administered IV) in infants with congenital hyperinsulinism (CHI). Patients <1 year of age with CHI that requires glucose infusion to prevent hypoglycemia and that are non-responsive to diazoxide. The patient will be given a randomized, blinded 48-hour continuous infusion treatment that will compare the glucose infusion rate (GIR) response between glucagon and placebo. This study will evaluate the effect of exogenous glucagon administered by continuous subcutaneously infusion via patch-pump (e.g. OmniPod) by measuring the rate of glucose that must be infused to maintain the blood sugar in the euglycemic range (>70 mg/dL). The lower the GIR, the greater the effect of the exogenous glucagon.

In the clinical trial, half the subjects are given placebo and the other half CSI glucagon during a 2-day blinded phase, while continuing D50. Following the blinded phase, subjects are eligible for open-label CSI glucagon. The blind has not been broken (as of Jul. 14, 2018), but open-label results from one study subject treated to-date are available. The CSI glucagon administered in this study was a non-aqueous glucagon formulation with a peptide concentration of 5 mg/mL with 5% (w/v) trehalose dissolved in dimethyl sulfoxide (DMSO).

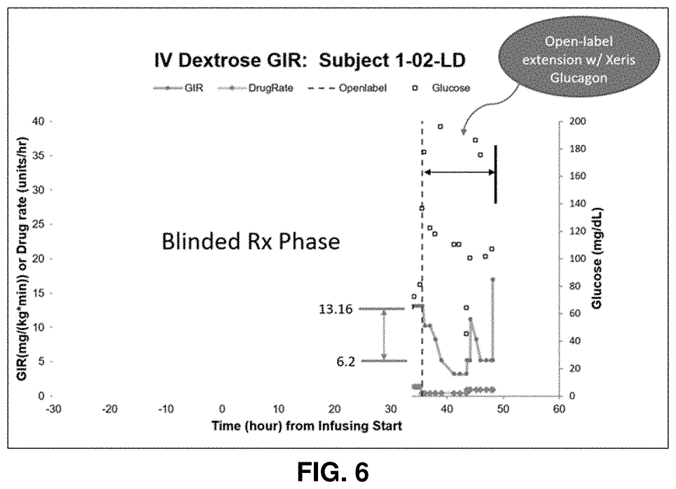

As shown in FIG. 6, an infant receiving CSI glucagon experienced a clinically meaningful 65% reduction in GIR, comparing the average level during the last 12 hours of the blinded phase and the last 12 hours of open-label treatment. During CSI-glucagon treatment, glucagon infusion rate (GIR) was reduced to an average of 6.2 mg/(kg*min), a level that would allow removal of the PICC line for long-term maintenance. No side effects or signs of intolerance were observed.

In summary, the data indicates a clinically significant reduction in the amount of glucose that must be infused to maintain the patient's glucose levels in the euglycemic range.

All of the compositions and/or methods disclosed and claimed herein can be made and executed without undue experimentation in light of the present disclosure. While the compositions and methods of this disclosure have been described in terms of some embodiments, it will be apparent to those of skill in the art that variations may be applied to the compositions and methods and in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of any invention as defined by the appended claims.

Embodiments of the present disclosure have been described in an illustrative manner, and it is to be understood that the particular embodiments depicted in the figures and the terminology which has been used has been intended in a nature of words of description rather than of limitation. It is to be further understood that any combination of the ingredients/therapeutic agents described in the foregoing paragraphs are deemed to be encompassed by the appended claims. It is to be further understood that all specific embodiments of the delivery apparatus are deemed to be encompassed by the appended claims. Many modifications and variations of the present disclosure are possible in light of the above teachings. It is therefore to be understood that the obvious modifications are deemed to be encompass within the appended claims.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this disclosure. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A method of treating congenital hyperinsulinism in a subject, the method comprising:
(a) parenterally administering to the subject a first composition comprising a glucagon, a glucagon analogue, or a salt form of either thereof; and
(b) optionally administering to the subject a second composition comprising glucose, a glucose analogue, or a salt form of either thereof,
wherein administration of the first composition sufficiently increases blood glucose level in the subject such that the second composition is not administered or the second composition is administered at a glucose infusion rate (GIR) of less than 20 mg/(kg*min).

2. The method of claim 1, wherein the second composition is administered to the patient.

3. The method of claim 2, wherein the GIR is less than 15, or less than 10 mg/(kg*min).

4. The method of claim 3, wherein the GIR is less than 8 mg/(kg*min).

5. The method of claim 2, wherein the GIR is at least 33% less than what would otherwise be needed had the subject not being administered the first composition.

6. The method of any one of claims 1 to 5, wherein the second composition is intravenously administered to the subject.

7. The method of any one of claims 1 to 6, wherein the subject is being treated with or has been previously treated with glucose injection at a second GIR prior to step (a), and wherein the second GIR is greater that the GIR.

8. The method of claim 6, wherein the glucose injection prior to step (a) is being or has been administered through a peripherally inserted central catheter.

9. The method of any one of claims 1 to 8, wherein the second composition is an aqueous composition comprising 5 w/w % to 60 w/w % d-glucose.

10. The method of claim 9, wherein the second composition comprises about 50 w/w % d-glucose.

11. The method of claim 9, wherein the second composition comprises about 10 w/w % d-glucose.

12. The method of any one of claims 1 to 11, wherein the first compositions is administered from a glucagon delivery apparatus.

13. The method of claim 12, wherein the glucagon delivery apparatus comprises:
a reservoir containing the first composition;
a sensor configured to measure a patient's blood glucose level; and
an electronic pump configured to intradermally, subcutaneously or intramuscularly deliver at least a portion of the composition to a patient based on the patient's measured blood glucose level.

14. The method of claim 13, where the sensor is configured to transmit data wirelessly, via radio frequency, or via a wired connection, to a processor configured to control operation of the electronic pump.

15. The method of claim 14, where the processor is configured to control operation of the pump based at least in part on the data obtained by the sensor.

16. The method of claim 15, where the processor is configured to control operation of the pump to intradermally, subcutaneously or intramuscularly inject at least a portion of the composition if the data obtained by the sensor indicates a glucose level below a defined threshold.

17. The method of any one of claims 12 to 16, further comprising a monitor configured to communicate information indicative of the patient's glucose level.

18. The method of claim 17, where the monitor comprises a speaker or a display device, or both.

19. The method of any one of claims 17 to 18, where the monitor is configured to communicate an alert when a glucose level of the patient is estimated to be at a defined threshold.

20. The method of any one of claims 12 to 19, where the apparatus is configured to allow manual adjustment of at least one of a delivery rate and a dose of the composition intradermally, subcutaneously or intramuscularly delivered by the pump.

21. The method of any one of claims 12 to 20, where the composition does not include a drug capable of decreasing the blood glucose level in the patient and/or where the apparatus is not capable of injecting a composition comprising a drug capable of decreasing the blood glucose level in the patient.

22. The method of any one of claims 12 to 22, where the composition further includes a drug capable of decreasing the blood glucose level in the patient and/or where the apparatus is capable of injecting a composition comprising a drug capable of decreasing the blood glucose level in the patient.

23. The method of any one of claims 21 to 22, where the drug capable of decreasing the blood glucose level in the patient is insulin, an insulin mimetic peptide, incretin, or an incretin mimetic peptide.

24. The method of any one of claims 12 to 23, wherein the apparatus is a closed-loop system for delivering glucagon to the patient.

25. The method of any one of claims 12 to 24, wherein the apparatus is an open-loop system for delivering glucagon to the patient.

26. The method of any one of claims 12 to 25, wherein the apparatus is a no-loop system for delivering glucagon to the patient.

27. The method of any one of claims 1 to 26, wherein the first composition is a single-phase solution comprising the glucagon, glucagon analogue, or a salt form of either thereof, dissolved in a non-aqueous solvent.

27. The method of claim 27, wherein the first composition comprises glucagon, glucagon analogue, or a salt form of either thereof solubilized in an aprotic polar solvent.

28. The method of claim 27, wherein the first composition further comprises an ionization stabilizing excipient, wherein (i) the glucagon, glucagon analogue, or salt thereof is dissolved in the aprotic solvent in an amount from about 0.1 mg/mL up to the solubility limit of the glucagon, glucagon analogue, or salt thereof, and (ii) the ionization stabilizing excipient is dissolved in the aprotic solvent in an amount to stabilize the ionization of the glucagon peptide or salt thereof.

29. The method of claim 28, wherein the ionization stabilizing excipient is at a concentration of 0.1 mM to less than 100 mM.

30. The method of claim 29, wherein the ionization stabilizing excipient is a mineral acid.

31. The method of claim 30, wherein the mineral acid is hydrochloric acid.

32. The method of claim 28, wherein the aprotic solvent is DMSO.

33. The method of claim 28, wherein the aprotic solvent is a deoxygenated aprotic solvent.

34. The method of claim 28, wherein the ionization stabilizing excipient is HCl and the aprotic solvent is DMSO.

35. The method of claim 28, wherein the composition has a moisture content of less than 10, 5, or 3%.

36. The method of claim 28, wherein the composition further comprises a preservative at less than 10, 5, or 3% w/v.

37. The method of claim 36, wherein the preservative is trehalose.

38. The method of claim 28, wherein the composition further comprises a sugar alcohol at less than 10, 5, or 3% w/v.

39. The method of claim 38, wherein the sugar alcohol is mannitol.

40. The method of claim 27, wherein the first composition further comprises a carbohydrate, an amphoteric molecule, and optionally an acid.

41. The method of claim 40, wherein the aprotic polar solvent is DMSO, the carbohydrate is trehalose, the amphoteric molecule is glycine, and the optional acid is hydrochloric acid.

42. The method of any one of claims 40 to 41, wherein the first composition comprises at least 80 wt. % of the aprotic polar solvent, 3 to 7 wt. % of the carbohydrate, 0.001 to 0.1 wt. % of the amphoteric molecule, and 0 wt. % to less than 0.1 wt. % of the acid.

43. The method of any one of claims 40 to 42, where the first composition comprises, consists essentially of, or consists of glucagon, the glucagon analogue, or the salt form of either thereof, the aprotic polar solvent, the amphoteric molecule, the carbohydrate, and optionally the acid.

44. The method of any one of claims 1 to 43, where the first composition has a water content of 0 to less than 15 wt. %, 0 to less than 3 wt. %, 3 to 10 wt. %, or 5 to 8 wt. %.

45. The method of any one of claims 1 to 44, where the glucagon, glucagon analogue, or salt form of either thereof, has been previously dried from a buffer, wherein the dried glucagon, glucagon analogue, or salt form of either thereof, has a first ionization profile that corresponds to an optimal stability and solubility for the glucagon, glucagon analogue, or salt form thereof, wherein the dried glucagon, glucagon analogue, or salt form of either thereof, is reconstituted into an aprotic polar solvent and has a second ionization profile in the aprotic polar solvent, and wherein the first and second ionization profiles are within 1 pH unit of one another.

46. The method of claim 45, where the first or second or both ionization profiles correspond to the ionization profile of glucagon when solubilized in an aqueous solution having a pH range of about 1 to 4 or 2 to 3.

47. The method of any one of claims 1 to 46, where the first composition is a two-phase mixture of a powder dispersed in a liquid that is a non-solvent to the solid, where the powder comprises the glucagon, glucagon analogue, or a salt form of either thereof, and where the liquid is a pharmaceutically acceptable carrier, where the powder is homogeneously contained within a pharmaceutically acceptable carrier.

48. The method of claim 47, where the first composition is a paste, slurry, or suspension.

49. The method of any one of claims 47 to 48, where the powder has a mean particle size ranging from 10 nanometers (0.01 microns) to about 100 microns, with no particles being larger than about 500 microns.

50. The method of any one of claims 1 to 49, where the first composition has been stored in the reservoir for at least 1, 2, 3, 4, 5, 6, 7, 14, 21, 30, 45, or 60 days.

51. The method of any one of claims 1 to 50, where the first composition remains stable after being stored for one month or 6 months or 12 months or 18 months at room temperature.

52. The method of any one of claims 1 to 51, further comprising administering a third composition to the subject, wherein the third composition comprises diazoxide or octreotide, or a combination thereof.

53. The method of claim 52, wherein the third composition is administered before administration of the first composition, preferably within 12, 11, 10, 9, 8, 7, 6, 5, 4, 3, 2, or 1 hour before administration of the first composition.

54. The method of claim 52, wherein the third composition is administered simultaneously with the first composition.

55. The method of claim 52, wherein the third composition is administered after administration of the first composition, preferably within 12, 11, 10, 9, 8, 7, 6, 5, 4, 3, 2, or 1 hour after administration of the first composition.

56. The method of any one of claims 1 to 55, wherein the subject is a human.

57. The method of claim 56, wherein the human is less than 20 years old, preferably less than 10 years old, more preferably less than 5 years old, or even more preferably within 0 to 3 years old, or within 0 to 12 months old.
